(19) United States
(12) Patent Application Publication
(10) Pub. No.: US 2025/0018220 A1
(43) Pub. Date: Jan. 16, 2025

(54) LASER APPLICATOR

(71) Applicant: Alma Lasers Ltd., Caesarea (IL)

(72) Inventors: Adi Caron, Pardesiya (IL); Miki Slonim, Binyamina (IL); Avner Lam, Givat Haim M. (IL)

(21) Appl. No.: 18/768,119

(22) Filed: Jul. 10, 2024

Related U.S. Application Data
(60) Provisional application No. 63/526,688, filed on Jul. 14, 2023.

(51) Int. Cl. A61N 5/067; A61N 5/06

(52) U.S. Cl. CPC A61N 5/067; A61N 5/0616; A61N 2005/0626; A61N 2005/0644; A61N 2005/0664

(57) ABSTRACT

A modular, removable laser applicator tip with wireless identification and with linear position control of optical elements, including lenses, splitters, and KTP crystals. The tip snaps into the applicator at a precise axial orientation. In splitter embodiments, a roller contact provides enhanced stability and cooling and maintains the laser beam at the optimal angle of incidence.

### RELATED APPLICATION

The present application claims the benefit of U.S. Provisional Patent Application No. 63/526,688 filed Jul. 14, 2023, the disclosure of which is incorporated herein by reference in its entirety.

### BACKGROUND

In surgical, medical, and aesthetic treatments, lasers are applied for heating the skin. Some treatments are vein ablation, scar alleviation, and hair removal.

The laser beam is applied from an applicator, held in contact with the skin. The applicator is connected to a console providing a user interface, power supply, a heat exchanger, and holders for storing the applicator. If the laser is small enough, it is built into the applicator, while a larger laser is sited in the console with its beam running through a mirrored arm to the applicator.

The laser beam passes through one or more optical elements in the tip of the applicator and out onto an area of incidence on the skin (the ‘spot’). The distance from the optical element to the skin is the working distance.

Examples of optical elements are a lens to refract the beam, a splitter to fractionate it into multiple beams, or a KTP crystal to convert the beam frequency. Optical elements can be combined, for example, a splitter followed by a lens.

The larger the spot produced, the lower the ‘fluence,’ which expresses joules of laser per square centimeter of skin.

The working distance of an optical element from the skin determines the spot size produced. For example, a lens at a relatively short working distance produces a small spot, with consequently high fluence, suited for vein ablation; a greater working distance increases the spot size, lowering the fluence, for tattoo removal; and a still greater working distance produces yet lower fluence, for skin rejuvenation.

To provide a range of spot sizes, or a variety of optical elements, a set of threaded tips is provided, each containing one or more optical elements set at a particular working distance.

To change the spot size or optical element, the device operator unscrews the tip and replaces it with one having the desired optical element and/or working distance. Drawbacks, in addition to the need for multiple tips, is that treatment is interrupted and there is automated tip detection, therefore a mismatch can occur, for example the operator sets the system for treatment A but mistakenly has installed the wrong tip.

Another drawback of a threaded tip is that the axial orientation when fully screwed in varies from one tip to another. As a result, for optical elements requiring specific axial orientation (e.g., splitter, crystal), the tip must be custom aligned for a specific applicator.

An alternative to swappable tips with optical elements at different working distances is an adjustable permanent tip. In one version, the operator twists the tip to move the optical element between working distance set points. However, the positioning mechanism is not precise, does not allow a continuous range of distances, and choice is limited to the built-in optical element.

Another type of adjustable permanent tip, with the same drawbacks, is described in Korean patent no. KR101824460: a lens moving device for moving a lens to adjust the irradiation point size of the laser.

In some cases, an automatic trigger for the laser is added in the form of a removable roller and associated rotation sensor. In use, the applicator is rolled along the skin and the sensor communicates rotation data to a controller, which triggers the laser at defined intervals. The result is a strip of skin with a spot (or spots in the case of a splitter optical element) laid down at defined intervals.

A drawback is that the roller does not provide good support for maintaining the applicator at the correct orientation to produce the best angle of incidence. Too small an angle puts the spot too close to the applicator and difficult to view, too large angle puts the spot too far away from the applicator and distorts the shape and size.

A further drawback is that the removable roller installation includes connecting a wire from the rotation sensor to the applicator. The wired connection adds an installation task and presents cleaning challenges.

Thus, there remains a considerable need for laser tip devices and methods to overcome the limitations described in this section.

### BRIEF SUMMARY

The present invention is directed to skin-treatment laser applicators with removable tips containing optical elements, where the tip is identified at installation and the linear position of the optical element is automatically adjustable.

Among the different possibilities contemplated, the optical element is positioned by a motorized cam.

It is further contemplated that insertion of the tip invokes homing and verification of freedom of travel for the optical element.

It is further contemplated that the tip identity serves as a key to set system operation parameter values and to position the optical element for a default treatment, which is presented, together with other possible treatments, for operator selection.

It is further contemplated that a removable roller and support arm may be provided so that the applicator can be rolled across the skin with the laser triggered at defined intervals and the applicator maintained at a preferred angle of incidence.

Various objects, features, aspects, and advantages of the present invention will become more apparent from the following detailed description of preferred embodiments of the invention, along with the accompanying drawings in which like numerals represent like components

### BRIEF DESCRIPTION OF THE DRAWINGS

An exemplary embodiment of the present invention is illustrated by way of example in the accompanying drawings in which like reference numbers indicate the same or similar elements and in which:

FIG. 1 depicts an embodiment of a device according to the teachings herein.

FIG. 2 depicts a sectioned side view of the device of FIG. 1

FIG. 3 is a magnified detail of FIG. 2 with some parts removed for clarity.

FIG. 4 depicts portions of a roller contact of an embodiment of a device in accordance with the teachings herein, oriented as in use.

FIG. 5a is a perspective view of the roller contact of FIG. 4 on its side.

FIG. 5b is a front view of the roller contact of FIG. 4 oriented as in use.

FIG. 5c is a side view of the roller contact of FIG. 4, oriented as in use.

FIG. 6 is a flow chart of a sequence for checking freedom of travel and homing an optical element of a device in accordance with the teachings herein.

### COMPONENT, REFERENCE NUMBER AND REPRESENTATIVE FIGURE

| Ref. no. | component | figure |
|---|---|---|
| 14 | tissue surface | 2 |
| 16 | laser | 2 |
| 18 | beam path axis | 2 |
| 20 | applicator | 1 |
| 22 | body | 1 |
| 24 | tip | 1 |
| 26 | platform | 2 |
| 28 | skin contact | 1 |
| 30 | positioning system | 2 |
| 32 | motor | 4 |
| 34 | optical element | 4 |
| 36 | controller | 4 |
| 38 | encoder | 4 |
| 40 | cam channel | 4 |
| 42 | cam follower | 4 |
| 44 | shaft | 4 |
| 46 | gear | 4 |
| 50 | rod | 3 |
| 52 | fixed channel | 3 |
| 54 | tip connector | 2 |
| 56 | identifier | 2 |
| 58 | identification detector | 2 |
| 60 | roller contact connector post | 5a |
| 62 | laser beam | 5b |
| 64 | line of travel | 5b |
| 66 | area of incidence | 5b |
| 68 | angle of incidence | 5c |
| 70 | roller contact | 5a |
| 72 | support | 5a |
| 74 | support contact surface | 4 |
| 76 | roller | 4 |
| 78 | rotation wheel | 4 |
| 80 | rotation sensor | 4 |
| 82 | thermoelectric cooler | 2 |
| 84 | fractional array of spots | 5b |

### DETAILED DESCRIPTION

The present invention represents several improvements in a laser applicator for surgical, medical, and aesthetic treatments of skin.

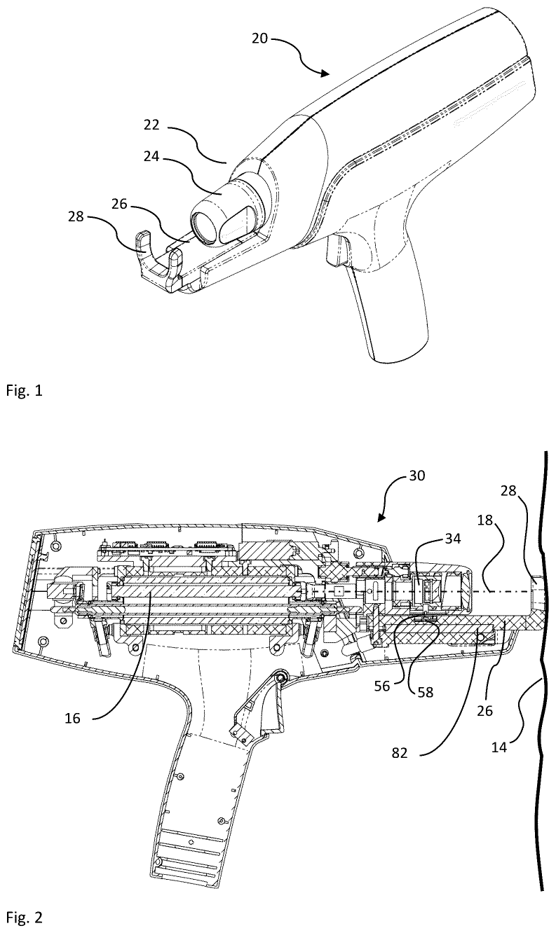

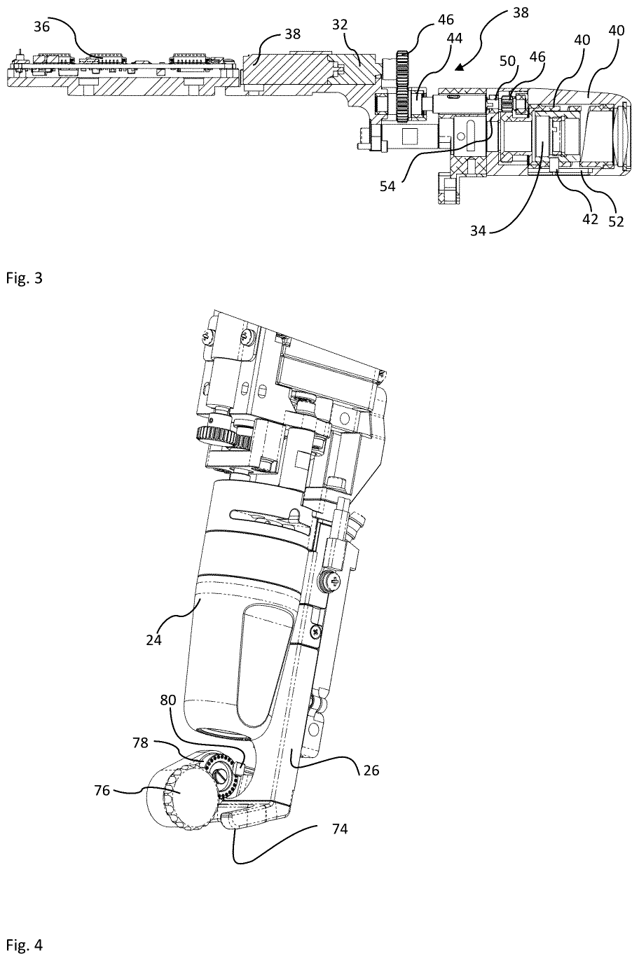

FIG. 1, FIG. 2, and FIG. 3 illustrate an embodiment of a laser applicator 20 in accordance with the teachings herein.

Tip 24, containing one or more optical elements 34, is adapted for easy installation and removal from the body 22 of the applicator. When the tip is installed, the optical element is on the optical path 18 of the beam of laser 16.

The optical element can be one or more of a lens to refract the beam, a splitter to fractionate it into multiple beams, a KTP crystal to convert the beam frequency, or a combination of one or more of these optical elements.

For installation of the tip on the body of the applicator, precise alignment is effected by mating of shaped posts (not shown) to corresponding receptacles (not shown) in the body and maintained by magnet pairs 54 in the body and the tip. An advantage of this snap-in arrangement for inserting and connecting the tip in the applicator is that it is fast and easy to swap tips and the tip axial orientation is uniform for any tip on any applicator, therefore custom axial calibration of the optical element is not required as it was for threaded tips.

A platform 26 extends from the body and terminates in a removable skin contact 28. When the applicator is in use, the skin contact provides maintains the outer surface of the optical element at a set working distance from the skin.

A thermoelectric cooler 82 in the platform conductively cools the skin contact and therefrom the skin. There is a small gap between the cooled platform and the tip to avoid condensation on the optical element.

The tip comprises an identifier magnet 56 that, when the tip is installed in the body of the applicator, lies opposite a Hall sensor identification detector 58 in the platform.

The applicator comprises a positioning system 30 for moving one or more of the optical elements linearly. The positioning system includes a motor 32 in the body that is controlled by the controller and paired with an encoder 38 that tracks motor rotation. The motor is geared 46 to a shaft 44 that terminates in a spring-loaded flat blade.

The tip comprises a rod 50 that is slotted at one end. When a tip is installed, the slotted end of the rod butts against the blade of shaft 44 and the identification detector signals the controller that a tip has been installed, causing the controller to rotate the motor till it is seated in the slot (step 108 below). The rod is geared at its other end to a cylinder comprising a helical cam channel 40. A cam follower 42 projects from the optical element through the cam channel 40 and into a linear fixed channel 52. In this arrangement, motor rotation will rotate the cam channel, pushing the cam follower along the fixed channel and thereby moving the optical element linearly, with the direction determined by that of the motor rotation.

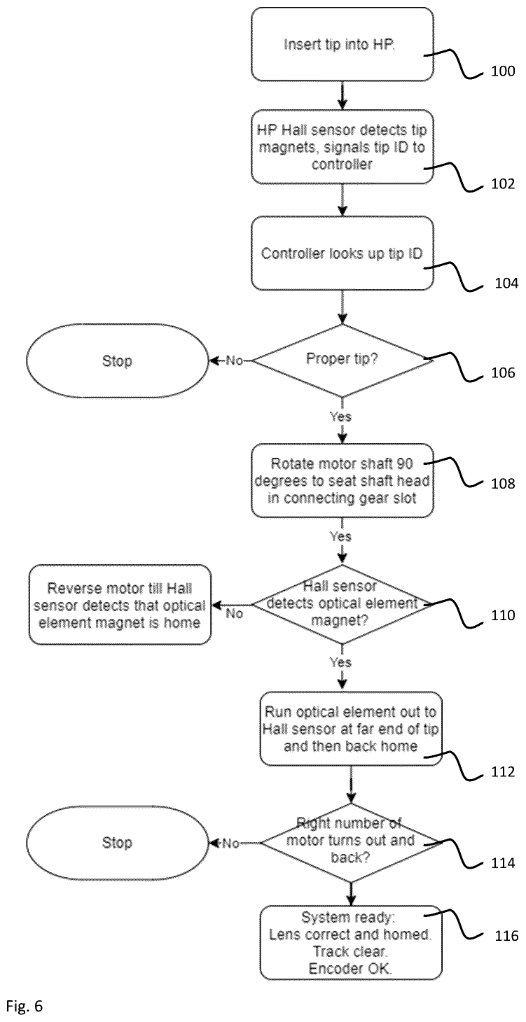

FIG. 6 summarizes what happens when the tip is inserted into the applicator.
100 The tip is inserted.
102 The identification detector reads the tip identification and sends the identification to the controller.
104, 106 The controller checks that the tip is proper for the treatment selected by an operator.
108 The motor rotates shaft 44 a quarter turn, to ensure that its blade aligns to the slot in the rod 50 at which point the spring load urges the blade into the slot. The motor is now connected through the components to the cam channel, such that when the motor rotates, the cam channel turns, pushing the cam follower in the fixed channel, back or forth, according to the direction of motor rotation.
110 The controller checks that a home magnetic sensor (58) has detected a magnet (56) attached to the optical element. If not, the motor is reversed until the magnet is detected, indicating that the optical element is homed.
112 The motor is activated to move the optical element out a defined number of motor rotations, then reversed the same number of rotations till the element magnet is detected by the home magnetic sensor.
114 The controller verifies that the encoder has read the same number of motor rotations for the trip out and the trip back of step 112. If not, a warning is issued.
116 If this point is reached, it indicates that the correct tip has been installed, the optical element is homed in the tip, the cam follower track is clear, and the encoder is working correctly.

The installed tip identification is used by a system control program to select operating parameter values for a default treatment and the optical element is moved to the working distance for that treatment.

The operator is presented with the option to approve selection of the default treatment or else to select from other treatments appropriate for the optical element in the tip.

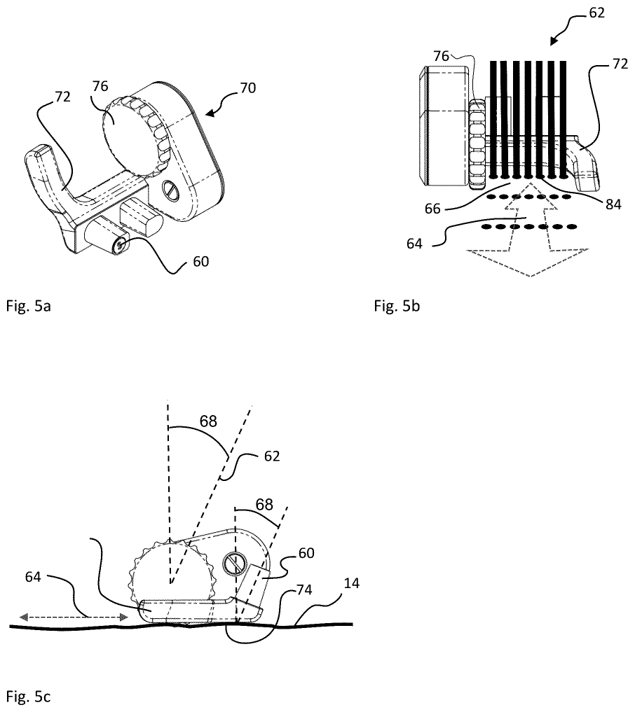

FIGS. 4, 5a, 5b, and 5c illustrate an alternative embodiment. The optical element can be any of the three types mentioned. In the case illustrated, the optical element is a splitter, which splits laser beam 62 into one or several rows of beams to apply a fractional array of spots 84 on the skin.

Skin contact 28 is a roller contact 70 with two projecting roller contact connector posts 60 that mate to receptacles in the platform 26, with one of the connectors comprising a magnet that pairs to a magnet in the platform. The roller contact enables the applicator to be rolled across the skin on roller 76 in a linear direction of travel 64. Each time that the roller has rotated a defined amount, the controller triggers the laser, applying an array of fractional spots 84. In this way, a series of separates arrays can be laid at defined intervals along a band of skin that can be as long as the roller has freedom to roll.

The roller 76 is geared to a rotation wheel 78, which comprises magnets around its perimeter. The magnets are detected by a rotation sensor 80 on the platform 26, providing a wireless reading of the rotation of the roller.

A support (72) extends extending perpendicularly from behind the roller and curves around to lie parallel to the roller. The support has a support contact surface (74) coplanar with the bottom of the roller, such that when the adapter is brought in contact with the skin, it is supported both by the roller and by the broad support contact surface, which both stabilizes the applicator and cools the skin.

The roller and the support define three sides of a roughly rectangular area of skin 66 on which the split laser beam 62 is incident when triggered, with the fourth, forward side, open. The angle between contact surface 74 and connector posts 60 sets the applicator at the optimum angle of incidence 68 for split laser beam 62. Formerly, these rolling fractionated lasers were not constrained around the axis of the roller, leaving it to the operator to try to maintain the laser at the best angle of incidence.

In some embodiments, the preferred angle of incidence is between 10 degrees and 40 degrees. In some embodiments, the preferred angle of incidence is between 15 degrees and 25 degrees. In some embodiments, the preferred angle of incidence is between 19 degrees and 21 degrees.

1. An applicator (20) for delivering a laser beam (16) to mammalian skin, the applicator comprising:
a. a removably connected tip (24) comprising:
i. at least one optical element (34) selected from the group consisting of a lens, a beam splitter, and a KTP crystal; and
ii. an identifier (56); and
b. an identification detector (58) adapted to detect the identifier upon connection of the tip to the applicator; and
a positioning system (30) adapted to position the optical element (34) along the laser path.

2. The applicator of claim 1 wherein the identifier is at least one magnet and the identification detector is a magnetic sensor.

3. The applicator of claim 1 wherein the removable connection of the tip to the applicator is by magnet (54).

4. The applicator of claim 1 wherein the positioning system comprises:
a. a motor (32) and associated encoder (38);
b. a controller (36) configured to receive motor rotation data from the encoder and to control the motor;
c. a drive shaft (44) geared at one end to the motor (32) and adapted at the other end to mate with a rod (50) in the tip;
d. the rod geared to a rotatable cam channel (40); and
e. a cam follower (42) on the optical element, extending through the cam channel and into an axial fixed channel (52);
whereby rotation of the motor is translated into linear movement of the optical element.

5. The applicator of claim 4 further adapted to home the optical element and to verify freedom of travel of the optical element, by
a. fully retracting the optical element to a home position; then
b. running the optical element out to a defined end point; then
c. running the optical element back to the home position.

6. The applicator of claim 1 further adapted to cooperate with a user-controlled system operation program to:
a. set system operation parameter values for a default treatment defined for the tip identifier;
b. position the optical element to the working distance for the treatment;
c. notify the user which treatment has been selected, and
d. offer the user the choice to approve the selected treatment or to switch to another treatment that is possible with the tip.

7. The applicator of claim 1 further comprising:
a. a removably connected roller contact (70), comprising:
i. a roller (76) adapted to be rolled across the tissue in a linear direction of travel (64);
ii. a rotation wheel (78) geared to the roller, comprising magnets arranged around its perimeter; and
iii. a support (72) extending perpendicular and parallel to the roller and having a support contact surface (74) coplanar with the bottom of the roller, wherein the support is adapted to support the applicator on the skin and to support maintaining the applicator a defined angle of incidence coplanar with the direction of travel; and
b. a magnetic rotation sensor (80) adapted to trigger the laser when the rotation wheel rotates a defined distance.

8. The applicator of claim 7 operable to be rolled along the skin, firing the laser at predefined distance intervals onto an area of skin (66) passing beneath an area bounded on one side by the roller, and on the back and other side by the support.

9. The applicator of claim 7 wherein the angle of incidence is between 10 degrees and 40 degrees.

10. The applicator of claim 7 wherein the angle of incidence is between 15 degrees and 25 degrees.

11. The applicator of claim 7 wherein the angle of incidence is between 19 degrees and 21 degrees.
